(19) United States
(12) Patent Application Publication
(10) Pub. No.: US 2014/0179808 A1
(43) Pub. Date: Jun. 26, 2014

(54) Detection of Ovarian Carcinoma by Assay for Autoantibodies to Multiple Antigens

(71) Applicant: BIO-RAD LABORATORIES, INC., HERCULES, CA (US)

(72) Inventor: John J. Flanagan, Walnut Creek, CA (US)

(73) Assignee: BIO-RAD LABORATORIES, INC., HERCULES, CA (US)

(21) Appl. No.: 14/060,472

(22) Filed: Oct. 22, 2013

Related U.S. Application Data
(60) Provisional application No. 61/717,320, filed on Oct. 23, 2012.

(51) Int. Cl. G01N 33/574

(52) U.S. Cl. CPC G01N 33/57449
USPC 514/789; 436/501

(57) ABSTRACT

Assays and reagents for the detection of autoantibodies to detect ovarian carcinomas are provided.

### CROSS-REFERENCE TO RELATED APPLICATIONS

This application claims priority benefit of U.S. provisional application No. 61/717,320, filed Oct. 23, 2013, which application is herein incorporated by reference.

### BACKGROUND OF THE INVENTION

Cancer Antigen 125 (CA-125) is currently the most widely used marker for ovarian carcinoma. However, even though as a marker CA-125 has high specificity for the disease, it lacks sensitivity and has only a low positive predictive value.

In order to increase the sensitivity of ovarian cancer detection while maintaining high specificity, additional markers are needed. Autoantibodies to tumor-associated antigens are attractive as candidates for cancer biomarkers, as they are present early in disease, are produced at a high level and are stable in blood. Multiple studies have used a non-biased screening approach to identify autoantibodies present in ovarian carcinoma using random peptide or protein arrays. Although autoantibodies have been identified in patient serum using this method, it is probable that most of the targets will fail as useful biomarker in clinical studies, as the autoantibodies are not likely to be cancer-specific, as the corresponding antigens have little or no known association with cancer pathways.

There is thus a need to increase the sensitivity of ovarian cancer detection. This invention addressed this need.

### REFERENCE TO A “SEQUENCE LISTING” SUBMITTED AS AN ASCII TEXT FILE

The Sequence Listing written in file—946-1.TXT, created on Oct. 22, 2013, 20,480 bytes, machine format IBM-PC, MS-Windows operating system, is hereby incorporated by reference in its entirety for all purposes.

### BRIEF SUMMARY OF THE INVENTION

The invention is based, in part, on the discovery of biomarkers for ovarian cancer that increase the sensitivity of ovarian cancer detection while maintain high specificity. Thus, in one aspect, the invention provides a method of determining the amount of autoantibodies to insulin-like growth factor binding protein 2 (IGF2BP2), HMGA2, MYCN, and TGIF2LX in a blood sample from a human patient that is being evaluated for ovarian cancer, the method comprising: contacting a blood sample obtained from the patient with one or more peptides of 10 amino acids or greater in length from each of the antigens IGF2BP2, HMGA2, MYCN, and TGIF2LX; and measuring the amount of selective binding of autoantibodies to the one or more peptides. In some embodiments, the method further comprises measuring the level of CA-125 in a blood sample from the patient. In some embodiments, the peptides are attached to a solid support. The peptides may be linked to the same solid support or each peptide may be linked to a different solid support. In some embodiments, each peptide is linked to a different bead, wherein each bead is distinguishable by flow cytometry. In some embodiments, the peptides from each antigen are linked to the same solid support and the peptides from the different antigens are linked to different solid supports. In some embodiments, the peptides comprise SEQ ID NOS:1-22. In some embodiments, specific binding of the autoantibody to the one or more peptides is detected by detecting binding of an anti-human IgG antibody to the autoantibody. In some embodiments, a method of the invention further comprises administering an agent to treat ovarian cancer when the patient has: a CA-125 level over about 100 IU/mL; or a CA-125 level between about 35 and 100 IU/mL and an autoantibody that specifically binds to IGF2BP2, HMGA2, MYCN, or TGIF2LX, or a fragment thereof; or a negative CA-125 result and an autoantibody that specifically binds to IGF2BP2, HMGA2, MYCN, or TGIF2LX, or a fragment thereof.

In a further aspect, the invention provides a kit for detecting ovarian cancer in a human patient, wherein the kit comprises the following components: one or more IGF2BP2 peptides that specifically detect an autoantibody to IGF2BP2; one or more HMGA2 peptides that specifically detect an autoantibody to HMGA2; one or more TGIF2LX peptides that specifically detect an autoantibody to TGIF2LX; and one or more MYCN peptides that specifically detect an autoantibody to MYCN. In some embodiments, the kit further comprises an anti-CA-125 antibody component. In some embodiments, the kit comprises an IGF2B2 peptide comprising the IGF2B2 protein sequence set forth in SEQ ID NO:2, an IGF2B2 peptide comprising the sequence set forth in SEQ ID NO:8, an IGF2B2 peptide comprising the sequence set forth in SEQ ID NO:9, an IGF2B2 peptide comprising the sequence set forth in SEQ ID NO:10 and an IGF2B2 peptide comprising the sequence set forth in SEQ ID NO:11. In some embodiments, the kit comprises an HMGA2 peptide comprising the HMGA2 protein sequence set forth in SEQ ID NO:1, an HMGA2 peptide comprising the sequence set forth in SEQ ID NO:5, an HMGA2 peptide comprising the sequence set forth in SEQ ID NO:6, and an HMGA2 peptide comprising the sequence set forth in SEQ ID NO:7. In some embodiments, the kit comprises a MYCN peptide comprising the MYCN protein sequence set forth in SEQ ID NO:3, a MYCN peptide comprising the sequence set forth in SEQ ID NO:12, a MYCN peptide comprising the sequence set forth in SEQ ID NO:13, a MYCN peptide comprising the sequence set forth in SEQ ID NO:14, a MYCN peptide comprising the sequence set forth in SEQ ID NO:15, a MYCN peptide comprising the sequence set forth in SEQ ID NO:16, and a MYCN peptide comprising the sequence set forth in SEQ ID NO:17. In some embodiments, the kit comprises a TGIF2LX peptide comprising the TGIF2LX protein sequence set forth in SEQ ID NO:4, a TGIF2LX peptide comprising the sequence set forth in SEQ ID NO:18, a TGIF2LX peptide comprising the sequence set forth in SEQ ID NO:19, a TGIF2LX peptide comprising the sequence set forth in SEQ ID NO:20, a TGIF2LX peptide comprising the sequence set forth in SEQ ID NO:21, and a TGIF2LX peptide comprising the sequence set forth in SEQ ID NO:22. In some embodiments, a kit of the invention at least two, three, four, five, six, seven, eight, nine, ten, eleven, twelve, thirteen, fourteen, fifteen, sixteen, seventeen, or all, of the following peptides: an IGF2BP2 peptide having the sequence of SEQ NO:8; an IGF2BP2 peptide having the sequence of SEQ ID NO:9; an IGF2BP2 peptide having the sequence of SEQ ID NO:10; an IGF2BP2 peptide having the sequence of SEQ ID NO 11; an HMGA2 peptide having the sequence of SEQ ID NO:5; an HMGA2 peptide having the sequence of SEQ ID NO:6; an HMGA2 peptide having the sequence of SEQ ID NO:7; a MYCN peptide having the of SEQ ID NO:12; a MYCN peptide having the of SEQ ID NO:13; a MYCN peptide having the of SEQ ID NO:14 a MYCN peptide having the of SEQ ID NO:15 a MYCN peptide having the of SEQ ID NO:16; a MYCN peptide having the of SEQ ID NO:17; a TGIF2LX peptide having the of SEQ ID NO:18; a TGIF2LX peptide having the of SEQ ID NO:19; a TGIF2LX peptide having the of SEQ ID NO:20; a TGIF2LX peptide having the of SEQ ID NO:21; and a TGIF2LX peptide having the of SEQ ID NO:22.

In some embodiments, the components of the kit, e.g., the peptide components, are linked to a solid support. In some embodiments, the components are individually linked to a different solid support. In some embodiments, the solid supports are beads. In some embodiments, the beads to which the components are individually linked are distinguishable from one another by flow cytometry. In some embodiments, the components are linked to the same solid support. In some embodiments, the kit further comprises an anti-human IgG antibody. In some embodiments, the anti-human IgG antibody is linked to a detectable label.

### DETAILED DESCRIPTION OF THE INVENTION

### I. Introduction

The present invention is based, in part, on the discovery that the presence of autoantibodies to insulin-like growth factor 2 mRNA binding protein 2 (IGF2BP2), High-mobility group AT-hook 2 (HMGA2), TGFB-induced factor homeobox 2-like, X-linked (TGIF2LX), and/or MYCN in the blood can be used as indicators for ovarian cancer. When used in combination with CA-125, detection of these autoantibodies provides for improved sensitivity for ovarian cancer detection relative to assays performed in which only CA-125 is detected.

Detection of IGF2BP2, HMGA2, TGIF2LX, and/or MYCN autoantibodies can be used in the diagnosis of ovarian cancer when the individual has negative or equivocal CA-125 levels. In some embodiments, where the level of CA-125 is equivocal, detection of IGF2BP2, HMGA2, TGIF2LX, and/or MYCN autoantibodies increases the specificity of ovarian cancer detection by reducing false positive for CA-125. In some embodiments where the CA-125 results are negative, detection of IGF2BP2, HMGA2, TGIF2LX, and/or MYCN autoantibodies increases the sensitivity of ovarian cancer detection.

The detection of ovarian cancer can additionally be improved by employing one or more specific peptides fragments from the IGF2BP2, HMGA2, TGIF2LX, and/or MYCN antigen that are specifically bound by the autoantibody. Peptide fragments are typically employed in the assays of the invention in combination with a full-length protein. Elevated levels of autoantibodies that bind to the immunogenic fragment of the antigen acts as a confirmation for elevated levels of autoantibodies bound to the full-length antigen. Alternatively, autoantibodies may be identified that bind to a peptide fragment, but that do not bind to the full length antigen. For example, for IGF2BP2, patients may have autoantibodies that bind to specific fragments of IGF2BP2 as described herein, but not to the full-length protein. Accordingly, use of such fragments allow for identification of patients that would otherwise be classified as negative if only the full-length protein is employed.

### II. Autoantibodies that Specifically Bind IGFBP2, TGIF2LX, HMGA2, and/or MYCN in Ovarian Cancer Detection

As noted above, it has been discovered that autoantibodies to the antigens IGF2BP2, HMGA2, TGIF2LX, and/or MYCN can be used in conjunction with CA-125, thereby increasing the specificity of ovarian cancer detection. Studies of CA-125 have reported that at a cutoff value of 100 U/mL, the CA-125 specificity is 99.9%, meaning one false positive result per 1000 results in a healthy population (see, e.g., Bon, G. G., et al., Am. J. Obstet. Gynecol. 174:107-14 (1996); Skates, S J, et al., J. Clin. Oncol. 21:206s-210s (2003)). Further, CA-125 has a specificity of at most 98% at the commonly used cut off of 35 IU/mL. Accordingly, for every 100 healthy results essentially 2 out of 100 will be >35 but only 1/1000 will be over 100. Given low disease prevalence, and the fact that most ovarian cancer patients have CA-125>100 when diagnosed, many patients with serum values between 35-100 IU/mL will turn out to be false positives.

Detection of autoantibodies to IGF2BP2, HMGA2, TGIF2LX, and/or MYCN antigens in combination with CA-125 improves ovarian cancer detection, especially for patients who have somewhat elevated CA-125 levels (e.g., percentile levels at about 98-99.9 of normal values) of CA-125.

In some embodiments, detection of the presence or absence of ovarian cancer in an individual comprises detection of CA-125 and autoantibodies to one or more of the antigens IGF2BP2, HMGA2, TGIF2LX, and/or MYCN, wherein any of the following results indicate the presence of ovarian cancer:

CA-125 levels over 100 IU/ml regardless of the levels of autoantibodies to IGF2BP2, HMGA2, TGIF2LX, and/or MYCN autoantibodies; or

negative CA-125 levels or levels between 35-100 IU/ml and detection of autoantibodies that bind to full-length IGF2BP2, HMGA2, TGIF2LX, and/or MYCN antigens, or to a fragment of IGFBP2, HMGA2, TGIF2LX, or MYCN.

### Detection of CA-125

CA-125 can be detected in any format known in the art. Detection of CA-125 refers to detection of the intact CA-125 protein, or fragments thereof that are indicative of the presence of the intact CA-125 protein. A number of formats for detection of CA-125 can be used according to the invention. Generally, a capture agent, immobilized on a solid support, is used to capture CA-125 from the sample. The capture agent can be, for example, an antibody. Alternatively, the capture agent is a non-antibody protein. A large number of scaffolds for generating non-antibody proteins with high binding specificities are known or can be generated. See, e.g., Bestes, et al., Prof Natl Acad Sci USA. 96(5): 1898-1903 (1999); U.S. Pat. No. 7,115,396; U.S. Pat. No. 7,018,801; and US Patent Publication No. 2005/0221384. Once captured from a sample, CA-125 can be detected using a detection agent. The detection agent can be, for example, an antibody or non-antibody protein that specifically binds CA-125. The detection agent can be directly labeled (e.g., with a fluorescent or other label) or can be detected indirectly, e.g., via a secondary antibody that is detectably labeled, or by enzymatic reaction in embodiments where an enzyme (e.g., HRP) is linked to the detection or secondary antibody, or via affinity linkers such as biotin/streptavidin to link the detection reagent to the label.

In some embodiments, CA-125 in a sample is initially captured by contacting the sample with the capture agent immobilized on a solid support under conditions to allow for binding of CA-125, if present in the sample, to the immobilized capture agent. The presence of the captured CA-125 is then detected, optionally following one or more wash step to remove non-binding components of the sample.

In some embodiments, the solid support is a bead or particle (used interchangeably herein). Exemplary beads include but are not limited to those that can be sorted by flow cytometry, e.g., Luminex beads. Once CA-125 in the sample is captured, the particles are recovered and separated from some or all of the remaining reagents in the mixture. For example, in some embodiments, the sample is removed from the particles by washing the particles in an appropriately buffered solution. Particles can be recovered by any method known in the art. In some cases, the particles are pelleted by centrifugation and the remaining sample (i.e., the supernatant) is removed from the particles. In some embodiments, the particles are responsive to a magnetic field and a magnetic field is applied such that the liquid in a sample is removed while the particles adhere to a reaction vessel wall, separating the remaining liquid from the particles. The particles can optionally be washed, e.g., one or more times with an appropriate buffer, if desired.

The captured CA-125 is subsequently detected and quantified. In some embodiments, the CA-125 can be detected by incubating the captured CA-125 with a labeled antibody or non-antibody protein that specifically binds to CA-125, thereby allowing the labeled antibody to bind to the captured CA-125. Excess labeled antibody can be subsequently removed, and the remaining labeled antibody (associated with the particles) is detected and optionally quantified. The presence and quantity of the label can be used to estimate the amount of CA-125 in the original sample, for example, by comparing the quantity of label to a calibration curve based on known amounts of CA-125, as is well known in the art.

Alternative methods for detecting CA-125 can also be used. Without intending to limit the invention to a particular method of detecting CA-125, one alternative is a competition assay. In these embodiments, CA-125 immobilized on a solid support (e.g., a particle) is incubated with a sample as well as an exogenous CA-125 that is optionally labeled, thus allowing for competition of the exogenous CA-125 with any endogenous CA-125 in the sample. Reduction in signal from the label associated with the exogenous CA-125 is thus related to increased amount of endogenous exogenous CA-125 in the sample.

Detection of Autoantibodies to IGF2BP2, HMGA2, TGIF2LX, and/or MYCN

Autoantibodies to IGF2BP2, HMGA2, TGIF2LX, and/or MYCN markers can be detected using any format known in the art. In some embodiments, a capture agent, immobilized on a solid support, is used to capture the autoantibodies. The capture agent is typically a IGF2BP2, HMGA2, TGIF2LX, and/or MYCN peptide. The IGF2BP2, HMGA2, TGIF2LX, and/or MYCN peptide can be full-length or a fragment that comprises an epitope recognized by the autoantibody to be detected. As described below in more detail, in some embodiments, a full length antigen and a fragment of the antigen are separately used to capture the autoantibodies. In some embodiments, the fragments are at least, e.g., 10, 12, 15, 20, 25, 30, 40, 50 or more contiguous amino acids of the full length antigen. Alternatively, the capture agent can be an antibody that binds human IgG to capture autoantibodies that can then be detected using an IGF2BP2, HMGA2, TGIF2LX, and/or MYCN peptide. Thus, the detection agent in assays for autoantibodies can be, for example, (1) an IGF2BP2, HMGA2, TGIF2LX, and/or MYCN peptide; or (2) antibody that binds human IgG, depending on whether the peptide or human IgG was used in the capture step.

In some embodiments, autoantibodies in a sample are initially captured by contacting the sample with an IGF2BP2, HMGA2, TGIF2LX, and/or MYCN peptide immobilized on the solid support under conditions to allow for binding of the autoantibodies, if present in the sample, to the immobilized peptide. The presence of the captured autoantibodies is then detected. The IGF2BP2, HMGA2, TGIF2LX, and/or MYCN peptide can be linked directly to the solid support or can be linked indirectly via a linker. The linkage can be covalent or non-covalent (e.g., via biotin/streptavin affinity or the like).

In some embodiments, the solid support is a bead or particle. Exemplary beads include but are not limited to beads (particles) that can be sorted by flow cytometry, including but not limited to, Luminex beads. In some embodiments, the IGF2BP2, HMGA2, TGIF2LX, and/or MYCN peptides are linked to different beads, optionally different beads that can be sorted by flow cytometry. Once autoantibodies in the sample are captured, the particles are recovered and separated from some or all of the remaining reagents in the mixture. For example, in some embodiments, the sample is removed from the particles by washing the particles in an appropriately buffered solution. Particles can be recovered by any method known in the art. In some cases, the particles are pelleted by centrifugation and the remaining sample (i.e., the supernatant) is removed from the particles. In some embodiments, the particles are responsive to a magnetic field and a magnetic field is applied such that the liquid in a sample is removed while the particles adhere to a reaction vessel wall, separating the remaining liquid from the particles. The particles can optionally be washed, e.g., one or more times with an appropriate buffer, if desired.

The captured autoantibodies are subsequently detected and optionally quantified. In embodiments, the autoantibodies can be detected by incubating the captured autoantibodies with a labeled antibody that specifically binds to human IgG. Excess labeled antibody is subsequently removed, and the remaining labeled antibody (now associated with the particles) is detected and optionally quantified. The presence and quantity of the label can be used to estimate the amount of autoantibodies in the original sample, for example, by comparing the quantity of label to a calibration curve based on known amounts of autoantibodies as is well known in the art.

Any type of anti-human IgG antibody can be used in the assay for detection of autoantibodies. Anti-human antibodies can be generated by administering human IgG, optionally with an adjuvant, to a non-human animal thereby stimulating production of antibodies in the animal that bind to human IgG. Optionally, anti-human IgG antibodies can be generated in vitro, e.g., by screening phage display antibody libraries or other antibody libraries. The anti-human IgG antibodies can be for example, mouse, rat, rabbit, goat, donkey or other non-human animal antibodies.

As noted above, full-length IGF2BP2, HMGA2, TGIF2LX, and/or MYCN, and fragments of IGF2BP2, HMGA2, TGIF2LX, and/or MYCN that comprises an epitope recognized by an autoantibody, can be used as separate capture agents, or optionally, as separate detection agents. In these aspects, a full length antigen as well as one or more immunogenic fragment of the antigen are typically employed such that the amount of autoantibodies binding to the full length antigen can be differentiated from the amount of autoantibodies binding a particular fragment. While one fragment can be used in this aspect, in some embodiments, two or more fragments are separately used to detect the autoantibodies, and in some embodiments, the amount of autoantibodies binding each fragment is separately detectable. For example, in some embodiments, the full length antigen is linked to a first solid support and the fragment is linked to a second solid support such that the two solid supports can be distinguished. In some embodiments, the antigen and the fragment are linked to separate types of beads that can be separated based on mass, fluorescence, or other characteristics, thereby allowing for separate detection of autoantibodies binding thereto.

In some embodiments, assays to detect IGF2BP2, HMGA2, TGIF2LX, and/or MYCN autoantibodies employ full-length IGF2BP2, HMGA2, TGIF2LX, and/or MYCN protein sequences as shown in SEQ ID NO:1 (HMGA2), SEQ ID NO:2 (IGFBP2), SEQ ID NO:3 (MYCN), and SEQ ID NO:4 (TGIF2LX). In some embodiments, autoantibodies can be detected using one or more fragments that comprise the following amino acid sequences:

HMGA2:
(SEQ ID NO: 5)
MSARGEGAGQPSTSAQGQPA
HMGA2:
(SEQ ID NO: 6)
SAQGQPAAPAPQKRGRGRPR
HMGA2:
(SEQ ID NO: 7)
KKAEATGEKRPRGRPRKWDN
IGF2BP2:
(SEQ ID NO: 8)
MMNKLYIGNLSPAVTADDLR
IGF2BP2:
(SEQ ID NO: 9)
LRSRKIQIRNIPPHLQWEVL
IGF2BP2:
(SEQ ID NO: 10)
QFENYSFKISYIPDEEVSSP
IGF2BP2:
(SEQ ID NO: 11)
TVKGTVEACASAEIEIMKKL
MYCN:
(SEQ ID NO: 12)
CKNPDLEFDSLQPCFYPDED
MYCN:
(SEQ ID NO: 13)
GFAEHSSEPPSWVTEMLLEN
MYCN:
(SEQ ID NO: 14)
RAGAALPAELAHPAAECVDP
MYCN:
(SEQ ID NO: 15)
PVNKREPAPVPAAPASAPAA
MYCN:
(SEQ ID NO: 16)
SEASPRPLKSVIPPKAKSLS
MYCN:
(SEQ ID NO: 17)
EKLQARQQQLLKKIEHARTC
TGIF2LX:
(SEQ ID NO: 18)
TGRVLALPEHKKKRKGNLPA
TGIF2LX:
(SEQ ID NO: 19)
RRRILPDMLQQRRNDPIIGH
TGIF2LX:
(SEQ ID NO: 20)
AKSGPSGPDNVQSLPLWPLP
TGIF2LX:
(SEQ ID NO: 21)
LPLWPLPKGQMSREKQPDPE
TGIF2LX:
(SEQ ID NO: 22)
LTGIAQPKKKVKVSVTSPSS

Other peptide fragments that specifically bind to IGF2BP2, HMGA2, TGIF2LX, or MYCN autoantibodies can also be used. Peptide epitopes can be identified by epitope mapping. One approach is to synthesize overlapping peptides, for example 20 residues in length, with a six residue overlap, which cover the entire primary sequence of a protein. However, depending on the position of the epitope in the sequence, it is often desirable to use different length peptide sequences to best define the minimal epitope present in a protein, and to ensure that an epitope is not missed because it was artificially split between overlapping peptides.

In some embodiments, the peptides are 20 amino acids in length or greater, for example, 21, 22, 23, 24, 25, 26, 27, 28, 29, or 30 or more amino acids in length. In some embodiments, the peptides are in the range of from 20 amino acids to 50 amino acids in length, e.g., 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length. In some embodiments, fragments may be joined together, or modified to include additional amino acids and the N-terminus or C-terminus. In some embodiments, the sequence is extended on the N and/or C terminals to provide additional amino acid residues that are present in the flanking sequences in the protein. This can more closely mimic the primary, and to a certain extent, the secondary structure environment of the epitope. Additionally, residues including but not limited to one or more glycines or gamma amino butyric acid, can be appended to either terminus to provide a spacer to minimize steric interactions with, for example, a solid phase used in an immunoassay. Spacer length is often varied to determine empirically the best structure.

Because of the variable nature of the epitope and the potential effects due to the flanking sequences, in some embodiments, one can use peptides that vary in length by extending the N or C terminals by a certain number of residues. Another approach utilizes repeating peptide epitopes, or alternating epitopes with intervening spacer residues. The length of these peptides is often varied according to the number of repeating units desired.

Peptides can vary greatly in their chemical properties, particularly in regard to hydrophobicity and ionic nature. For example, in order to modulate the properties of a highly hydrophobic epitope, neutral and hydrophilic residues can be added to one or both termini. This will result in a more hydrophilic, and thus accessible epitope for antibody binding, and a generally more soluble peptide.

In some embodiments, peptides derived from hydrophobic regions of a protein can interact strongly with the surface of a bead to which they are coupled due to hydrophobic or other interactions. Ionic interactions of charged peptides with a bead surface can also occur. This can result in the inaccessibility or diminished binding of a peptide to antibodies that would typically be able to bind to it in the context of the native protein.

To overcome the undesirable interactions of peptides with solid phase supports used in immunoassays, the peptides can be modified in several ways. One way is to substitute hydrophobic residues in the peptide with hydrophilic ones, in order to reduce or minimize the hydrophobic interactions, and increased peptide accessibility. Similarly, charged peptide residues can be substituted with noncharged residues to eliminate ionic interactions with the solid phase. Accordingly, in some embodiments, the “antigens” used in the assay are not exactly fragments of the full-length antigen sequence, but instead are highly similar fragments, i.e., having at least two sequences of at least 3 or 4 amino acids that are identical to the full length antigen sequence, linked by one or two amino acids that correspond to a position in the full-length antigen, but is different from the amino acid at that position in the full-length antigen.

Additionally, residues in the peptide can be substituted with different residues which can improve the immunoreactivity of the peptide relative to the native structure. The amino acid residues that can be substituted, such as proline, typically result in a peptide with less freedom of movement or rotation, although, in many cases, the amino acids for substitution that provide optimal immunoreactivity must be determined empirically, or in some cases using molecular modeling. In some embodiments, non-natural amino acids are substituted for natural amino acids. Peptides can be modified by adding spacer groups of a variety of structures to position the peptide epitope further from the solid phase and minimize steric hindrance.

Peptides can be synthesized to reflect post translation modifications that are present in the native protein. Modifications include but are not limited to phosphorylation, glycosylation, cyclization, citrullinization, etc. to mimic the form present in the native molecule, particularly at a specific site in the protein.

Peptides may also be cyclized in several manners, such as via disulfide or amide bond formation, which provides a more rigid structure, and a more favorable binding epitope for antibodies.

Peptides can be produced using methods well known in the art including both chemical synthesis and recombinant production techniques.

### III. Methods of Detection

In some embodiments, the methods comprise the combined detection of CA-125 and detection of IGF2BP2, HMGA2, TGIF2LX, or MYCN autoantibodies. In some embodiments, each component to be detected is captured onto a different solid support. For example, in some embodiments, the assay involves a first solid support linked to a capture agent for CA-125, a second solid support linked to a capture agent for an IGF2BP2 antibody, a third solid support linked to a capture agent for HMGA2, a fourth solid support linked to a capture agent for MYCN, and a fifth solid supported linked to a capture agent for TGIF2LX. As explained above, in addition, there can be fragments of IGF2BP2, HMGA2, TGIF2LX, or MYCN that are also employed, each on separate solid supports, to separately capture and detect autoantibodies.

Alternatively, the assay can be designed such that capture agents or more than one component are linked to the same solid support. The presence, absence, or level of each component is determined by using different labels to detect the specific binding between the detection agent for each component. For example, in some embodiments, a first solid support (e.g., a bead) is linked to both a capture agent for CA-125 and a capture agent for an IGF2BP2, HMGA2, TGIF2LX, or MYCN autoantibody. This solid support is then contacted to a biological sample such that CA-125 or autoantibody binds their respective capture agents and the remaining sample is washed away. The specific, differently-labeled, detection agents are applied, thereby allowing quantitative detection of both CA-125 and the autoantibody using one solid support/reaction. Similarly, where a sample is evaluated for IGF2BP2, HMGA2, TGIF2LX, and MYCN, the IGF2BP2, HMGA2, TGIF2LX, and MYCN peptides can be linked to one solid support.

In some embodiments, different particles can be distinguished by flow cytometry by a characteristic independent of the presence or absence of the component to be detected (e.g., independent of CA-125 or IGF2BP2, HMGA2, and/or MYCN autoantibodies) on the respective particles. In these embodiments, the particles can be sorted and the amount of label associated with each particle can be determined, thereby allowing for simultaneous determination of the amount of different components from the sample on different particles.

A patient is considered positive for the presence of autoantibodies to IGF2BP2, HMGA2, TGIF2LX, and/or MYCN if the assay detects levels above threshold values.

One can correlate the results of the assay to the presence of ovarian cancer using cut-off values (also referred to as threshold values). Where a component of the sample is higher than a set cut-off value, the sample is “positive” for that component, which is indicative of ovarian cancer. In some embodiments, the threshold value distinguishes between one diagnosis and another. For example, a threshold value can represent the level of a component generally found to distinguish between cancer samples and normal samples with a desired level of sensitivity and specificity. Cut-offs can be, for example, those values above the 95^{th}, 98^{th}, 99^{th},99.9 or other percentile of healthy values.

In some embodiments, the threshold value can vary depending on the assays used to measure a component. Comparisons between a level of a component in a sample and a threshold value can be performed in any way known in the art. For example, a manual comparison can be made or a computer can compare and analyze the values to correlate to the likely presence of ovarian cancer.

While particular cut-off values are set forth above, it will be understood that other cut-off values can be established depending on how the correlation is established. In some embodiments, an algorithm is used to establish cut-off values and/or to correlate the patient data to prediction of the presence or absence of ovarian cancer in the subject. Algorithmic techniques for relating biomarkers of the present disclosure include but are not limited to a linear regression technique, a nonlinear regression technique, an ANOVA technique, a neural network technique, a genetic algorithm technique, a support vector machine technique, a tree learning technique, a nonparametric statistical technique, a forward, backward, and/or forward-backward technique, and a Bayesian technique. The word “technique” is intended to encompass a process in which a predictor is built by using patient exemplar pairs of biomarkers and phenotypes, and then refining such predictor algorithm in an iterative process by testing a version of the algorithm on unseen (“test”) data and making changes to mathematical coefficients of such algorithm in such a way to increase the accuracy and specificity of the predictor algorithm.

In some embodiments, the methods comprise recording a diagnosis, prognosis, risk assessment or classification, based on the level of components determined from an individual. Any type of recordation is contemplated, including but not limited to electronic recordation, e.g., by a computer.

This invention is applicable to the analysis of sample biological fluids, including but not limited to, physiological fluids such as whole blood, plasma, serum, urine, and saliva. In typical embodiments, the sample that is evaluated for autoantibodies and CA-125 is from blood, e.g., plasma or serum.

### V. Detectable Labels

The labels used can be any label that is capable of directly or indirectly emitting or generating detectable signal. In some embodiments, the labels are fluorophores. As noted in more detail below, if desired, fluorophores may also be incorporated into the particles themselves to distinguish one group of particles from another. A vast array of fluorophores are reported in the literature and thus known to those skilled in the art, and many are readily available from commercial suppliers to the biotechnology industry. Literature sources for fluorophores include Cardullo et al., Proc. Natl. Acad. Sci. USA 85: 8790-8794 (1988); Dexter, D. L., J of Chemical Physics 21: 836-850 (1953); Hochstrasser et al., Biophysical Chemistry 45: 133-141 (1992); Selvin, P., Methods in Enzymology 246: 300-334 (1995); Steinberg, I. Ann. Rev. Biochem., 40: 83-114 (1971); Stryer, L. Ann. Rev. Biochem., 47: 819-846 (1978); Wang et al., Tetrahedron Letters 31: 6493-6496 (1990); Wang et al., Anal. Chem. 67: 1197-1203 (1995).

The following is a list of examples of fluorophores:
4-acetamido-4′-isothiocyanatostilbene-2,2′ disulfonic acid
acridine
acridine isothiocyanate
5-(2′-aminoethyl)aminonaphthalene-1-sulfonic acid (EDANS)
4-amino-N-[3-vinylsulfonyl)phenyl]naphthalimide-3,5 disulfonate
N-(4-anilino-1-naphthyl)maleimide
anthranilamide
BODIPY
Brilliant Yellow
coumarin
7-amino-4-methylcoumarin (AMC, Coumarin 120)
7-amino-4-trifluoromethylcoumarin (Coumaran 151)
cyanine dyes
cyanosine
4′,6-diaminidino-2-phenylindole (DAPI)
5′,5″-dibromopyrogallol-sulfonaphthalein (Bromopyrogallol Red)
7-diethylamino-3-(4′-isothiocyanatophenyl)-4-methylcoumarin
diethylenetriamine pentaacetate
4,4′-diisothiocyanatodihydro-stilbene-2,2′-disulfonic acid
4,4′-diisothiocyanatostilbene-2,2′-disulfonic acid
5-[dimethylamino]naphthalene-1-sulfonyl chloride (DNS, dansylchloride)
4-(4′-dimethylaminophenylazo)benzoic acid (DABCYL)
4-dimethylaminophenylazophenyl-4′-isothiocyanate (DABITC)
eosin
eosin isothiocyanate
erythrosin B
erythrosin isothiocyanate
ethidium
5-carboxyfluorescein (FAM)
5-(4,6-dichlorotriazin-2-yl)aminofluorescein (DTAF)
2′,7′-dimethoxy-4′5′-dichloro-6-carboxyfluorescein (JOE)
fluorescein
fluorescein isothiocyanate
fluorescamine
IR144
IR1446
Malachite Green isothiocyanate
4-methylumbelliferone
ortho cresolphthalein
nitrotyrosine
pararosaniline
Phenol Red
phycoerythrin (including but not limited to B and R types)
o-phthaldialdehyde
pyrene
pyrene butyrate
succinimidyl 1-pyrene butyrate
quantum dots
Reactive Red 4 (Cibacron™ Brilliant Red 3B-A)
6-carboxy-X-rhodamine (ROX)
6-carboxyrhodamine (R6G)
lissamine rhodamine B sulfonyl chloride rhodamine
rhodamine B
rhodamine 123
rhodamine X isothiocyanate
sulforhodamine B
sulforhodamine 101
sulfonyl chloride derivative of sulforhodamine 101 (Texas Red)
N,N,N′,N′-tetramethyl-6-carboxyrhodamine (TAMRA)
tetramethyl rhodamine
tetramethyl rhodamine isothiocyanate (TRITC)
riboflavin
rosolic acid
lanthanide chelate derivatives

If desired, the fluorophores (or other labels) can be used in combination, with a distinct label for each analyte. In some embodiments, however, a single label is used for all labeled binding members, the assays being differentiated solely by the differentiation parameter distinguishing the individual particle groups from each other.

The attachment of any of these fluorophores to the desired component described above to form assay reagents for use in the practice of this invention is achieved by conventional covalent bonding, using appropriate functional groups on the fluorophores and on the component. The recognition of such groups and the reactions to form the linkages will be readily apparent to those skilled in the art.

Methods of, and instrumentation for, flow cytometry are known in the art, and can be used in the practice of the present invention. Flow cytometry in general resides in the passage of a suspension of particles (or cells) in as a stream through a light beam and coupled to electro-optical sensors, in such a manner that only one particle at a time passes the region of the sensors. As each particle passes this region, the light beam is perturbed by the presence of the particle, and the resulting scattered and fluoresced light is detected. The optical signals are used by the instrumentation to identify the subgroup to which each particle belongs, along with the presence and amount of label, so that individual assay results are achieved. Descriptions of instrumentation and methods for flow cytometry are found in the literature. Examples are McHugh, “Flow Microsphere Immunoassay for the Quantitative and Simultaneous Detection of Multiple Soluble Analytes,” Methods in Cell Biology 42, Part B (Academic Press, 1994); McHugh et al., “Microsphere-Based Fluorescence Immunoassays Using Flow Cytometry Instrumentation,” Clinical Flow Cytometry, Bauer, K. D., et al., eds. (Baltimore, Md., USA: Williams and Williams, 1993), pp. 535-544; Lindmo et al., “Immunometric Assay Using Mixtures of Two Particle Types of Different Affinity,” J. Immunol. Meth. 126: 183-189 (1990); McHugh, “Flow Cytometry and the Application of Microsphere-Based Fluorescence Immunoassays,” Immunochemica 5: 116 (1991); Horan et al., “Fluid Phase Particle Fluorescence Analysis: Rheumatoid Factor Specificity Evaluated by Laser Flow Cytophotometry,” Immunoassays in the Clinical Laboratory, 185-189 (Liss 1979); Wilson et al., “A New Microsphere-Based Immunofluorescence Assay Using Flow Cytometry,” J. Immunol. Meth. 107: 225-230 (1988); Fulwyler et al., “Flow Microsphere Immunoassay for the Quantitative and Simultaneous Detection of Multiple Soluble Analytes,” Meth. Cell Biol. 33: 613-629 (1990); Coulter Electronics Inc., United Kingdom Patent No. 1,561,042 (published Feb. 13, 1980); and Steinkamp et al., Review of Scientific Instruments 44(9): 1301-1310 (1973).

Similarly, methods of and instrumentation for applying and removing a magnetic field as part of an assay are known to those skilled in the art and reported in the literature. Examples of literature reports are Forrest et al., U.S. Pat. No. 4,141,687 (Technicon Instruments Corporation, Feb. 27, 1979); Ithakissios, U.S. Pat. No. 4,115,534 (Minnesota Mining and Manufacturing Company, Sep. 19, 1978); Vlieger, A. M., et al., Analytical Biochemistry 205:1-7 (1992); Dudley, Journal of Clinical Immunoassay 14:77-82 (1991); and Smart, Journal of Clinical Immunoassay 15:246-251 (1992). All of the citations in this and the preceding paragraph are incorporated herein by reference.

### VI. Solid Supports

Any type of solid support can be used in the invention. In some embodiments, the solid support is suitable for use in an ELISA assay. In some embodiments, the solid support is spherical or near-spherical. In some embodiments, the particles used in the practice of this invention are microscopic in size and formed of a polymeric material. Polymers that will be useful as microparticles are those that are chemically inert relative to the components of the biological sample and to the assay reagents other than the binding member coatings that are affixed to the microparticle surface. Suitable microparticle materials will also have minimal autofluorescence, will be solid and insoluble in the sample and in any buffers, solvents, carriers, diluents, or suspending agents used in the assay, and will be capable of affixing to the appropriate coating material. Examples of suitable polymers are polystyrenes, polyesters, polyethers, polyolefins, polyalkylene oxides, polyamides, polyurethanes, polysaccharides, celluloses, and polyisoprenes. Crosslinking is useful in many polymers for imparting structural integrity and rigidity to the microparticle. The size range of the microparticles can vary. In some embodiments, the microparticles range in diameter from about 0.3 micrometers to about 100 micrometers, e.g., from about 0.5 micrometers to about 40 micrometers, e.g., from about 2 micrometers to about 10 micrometers.

To facilitate the particle recovery and washing steps of the assay, the particles preferably contain a magnetically responsive material, i.e., any material that responds to a magnetic field. Separation of the solid and liquid phases, either after incubation or after a washing step, is then achieved by imposing a magnetic field on the reaction vessel in which the suspension is incubated, causing the particles to adhere to the wall of the vessel and thereby permitting the liquid to be removed by decantation or aspiration. Magnetically responsive materials of interest in this invention include paramagnetic materials, ferromagnetic materials, ferrimagnetic materials, and metamagnetic materials. Examples, include, e.g., iron, nickel, and cobalt, as well as metal oxides such as Fe₃O₄, BaFe₁₂O₁₉, CoO, NiO, Mn₂O₃, Cr₂O₃, and CoMnP.

The magnetically responsive material can be dispersed throughout the polymer, applied as a coating on the polymer surface or as one of two or more coatings on the surface, or incorporated or affixed in any other manner that secures the material in to the particle. The quantity of magnetically responsive material in the particle is not critical and can vary over a wide range. The quantity can affect the density of the microparticle, however, and both the quantity and the particle size can affect the ease of maintaining the microparticle in suspension for purposes of achieving maximal contact between the liquid and solid phase and for facilitating flow cytometry. An excessive quantity of magnetically responsive material in the microparticles may produce autofluorescence at a level high enough to interfere with the assay results. Therefore, in some embodiments, the concentration of magnetically responsive material is low enough to minimize any autofluorescence emanating from the material. With these considerations in mind, the magnetically responsive material in a particle in accordance with this invention is, for example, from about 0.05% to about 75% by weight of the particle as a whole. In some embodiments, the weight percent range is from about 1% to about 50%, e.g., from about 2% to about 25%, e.g., from about 2% to about 8%.

Coating of the particle surface with the appropriate assay reagent can be achieved by electrostatic attraction, specific affinity interaction, hydrophobic interaction, or covalent bonding. The polymer can be derivatized with functional groups for covalent attachment of the assay reagents by conventional means, notably by the use of monomers that contain the functional groups, such monomers serving either as the sole monomer or as a co-monomer. Examples of suitable functional groups are amine groups (—NH₂), ammonium groups (—NH₃⁴ or —NR₃⁺), hydroxyl groups (—OH), carboxylic acid groups (—COOH), and isocyanate groups (—NCO). Useful monomers for introducing carboxylic acid groups into polyolefins, for example, are acrylic acid and methacrylic acid.

Linking groups can be used as a means of increasing the density of reactive groups on the particle surface and decreasing steric hindrance. This may increase the range and sensitivity of the assay. Linking groups can also be used as a means of adding specific types of reactive groups to the solid phase surface if needed to secure the particular coating materials of this invention.

The capture agents can be directly or indirectly linked to the solid support via a linking agent. The capture agent and solid support can be conjugated via a single linking agent or multiple linking agents. For example, the capture agent and solid support may be conjugated via a single multifunctional (e.g., bi-, tri-, or tetra-) linking agent or a pair of complementary linking agents. In some embodiments, the capture agent and solid support are conjugated via two, three, or more linking agents. Suitable linking agents include, e.g., functional groups, affinity agents, stabilizing groups, and combinations thereof.

In some embodiments, an affinity agent (e.g., agents that specifically binds to a ligand) is the linking agent. In these embodiments, for example, a first linking agent is bound to the capture agent and a second linking agent is bound to the solid support. Affinity agents include receptor-ligand pairs, antibody-antigen pairs and other binding partners such as streptavidin/avidin and biotin. In some embodiments, the first linking agent is biotin and the second linking agent is streptavidin or avidin. In some embodiments, the first linking agent is a hapten (e.g., fluorescein) and the second linking agent is an anti-hapten (e.g., anti-fluorescein) antibody.

Functional groups include monofunctional linkers comprising a reactive group as well as multifunctional crosslinkers comprising two or more reactive groups capable of forming a bond with two or more different functional targets (e.g., peptides, proteins, macromolecules, semiconductor nanocrystals, or substrate). In some embodiments, the multifunctional crosslinkers are heterobifunctional crosslinkers comprising two different reactive groups.

Suitable reactive groups include, e.g., thiol (—SH), carboxylate (COOH), carboxyl (—COOH), carbonyl, amine (NH₂), hydroxyl (—OH), aldehyde (—CHO), alcohol (ROH), ketone (R₂CO), active hydrogen, ester, sulfhydryl (SH), phosphate (—PO₃), or photoreactive moieties. Amine reactive groups include, e.g., isothiocyanates, isocyanates, acyl azides, NHS esters, sulfonyl chlorides, aldehydes and glyoxals, epoxides and oxiranes, carbonates, arylating agents, imidoesters, carbodiimides, and anhydrides. Thiol-reactive groups include, e.g., haloacetyl and alkyl halide derivates, maleimides, aziridines, acryloyl derivatives, arylating agents, and thiol-disulfides exchange reagents. Carboxylate reactive groups include, e.g., diazoalkanes and diazoacetyl compounds, such as carbonyldiimidazoles and carbodiimides. Hydroxyl reactive groups include, e.g., epoxides and oxiranes, carbonyldiimidazole, oxidation with periodate, N,N′-disuccinimidyl carbonate or N-hydroxylsuccimidyl chloroformate, enzymatic oxidation, alkyl halogens, and isocyanates. Aldehyde and ketone reactive groups include, e.g., hydrazine derivatives for Schiff base formation or reduction amination. Active hydrogen reactive groups include, e.g., diazonium derivatives for Mannich condensation and iodination reactions. Photoreactive groups include, e.g., aryl azides and halogenated aryl azides, benzophenones, diazo compounds, and diazirine derivatives.

Other suitable reactive groups and classes of reactions useful in practicing the present invention are generally those that are well known in the art of bioconjugate chemistry. Currently favored classes of reactions available with reactive chelates are those which proceed under relatively mild conditions. These include, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982.

In some embodiments, the functional group is a heterobifunctional crosslinker comprising two different reactive groups that contain heterocyclic rings that can interact with peptides and proteins. For example, heterobifunctional crosslinkers such as N-[γ-maleimidobutyryloxy]succinimide ester (GMBS) or succinimidyl 4-[N-maleimidomethyl]cyclohexane-1-carboxylate (SMCC) comprise an amine reactive group and a thiol-reactive group that can interact with amino and thiol groups within peptides or proteins. Additional combinations of reactive groups suitable for heterobifunctional crosslinkers include, for example, carbonyl and sulfhydryl reactive groups; amine and photoreactive groups; sulfhydryl and photoreactive groups; carbonyl and photoreactive groups; carboxylate and photoreactive groups; and arginine and photoreactive groups. Examples of suitable useful linking groups are polylysine, polyaspartic acid, polyglutamic acid and polyarginine. N-hydroxysuccinimide (NHS), CMC 1-cyclohexyl-3-(2-morpholinoethyl)carbodiimide (CMC), N-Hydroxybenzotriazole (HOBt), and/or other crosslinking agents may be used.

In some embodiments, care is taken to avoid the use of particles that exhibit high autofluorescence. Particles formed by conventional emulsion polymerization techniques from a wide variety of starting monomers are generally suitable since they exhibit at most a low level of autofluorescence. Conversely, particles that have been modified to increase their porosity and hence their surface area, i.e., those particles that are referred to in the literature as “macroporous” particles, are less desirable since they tend to exhibit high autofluorescence. A further consideration is that autofluorescence increases with increasing size and increasing percentage of divinylbenzene monomer.

Multiplexing with the use of microparticles in accordance with this invention can be achieved by designing each particle (i.e., the “first” particle, and the “second” particle, and if relevant, the “third” particle, and the “fourth” particle, etc.) to have a distinctive differentiation parameter, which renders that group distinguishable from the other groups by flow cytometry.

One example of a differentiation parameter is the particle diameter, the various particle groups being defined by nonoverlapping diameter subranges. The widths of the diameter subranges and the spacing between mean diameters of adjacent subranges are selected to permit differentiation of the subranges by flow cytometry, and will be readily apparent to those skilled in the use of and instrumentation for flow cytometry. In this specification, the term “mean diameter” refers to a number average diameter. In some embodiments, the subrange width is about ±5% CV or less of the mean diameter, where “CV” stands for “coefficient of variation” and is defined as the standard deviation of the particle diameter divided by the mean particle diameter, times 100 percent. The minimum spacing between mean diameters among the various subranges can vary depending on the microparticle size distribution, the ease of segregating microparticles by size for purposes of attaching different assay reagents, and the type and sensitivity of the flow cytometry equipment. In some embodiments, best results will be achieved when the mean diameters of different subranges are spaced apart by at least about 6% of the mean diameter of one of the subranges, e.g., at least about 8% of the mean diameter of one of the subranges, e.g., at least about 10% of the mean diameter of one of the subranges. In some embodiments, the standard deviation of the particle diameters within each subrange is less than one third of the separation of the mean diameters of adjacent subranges.

Another example of a differentiation parameter that can be used to distinguish among the various groups of particles is fluorescence. Differentiation is accomplished by incorporating one or more fluorescent materials in the particles, the fluorescent materials having different fluorescent emission spectra and being distinguishable on this basis.

Fluorescence can in fact be used both as a means of distinguishing the particle groups from each other and as a means of detection and quantification for the assay performed on the particles. The use of fluorescent materials with different emission spectra can serve as a means of distinguishing the particle groups from each other and also as a means of distinguishing the particle group's classification from the (e.g., fluorescent) assay reported signals. An example of a fluorescent substance that can be used as a means of distinguishing particle groups is fluorescein and an example of a substance that can be used for the assay detection is phycoerythrin. In the use of this example, different particle groups can be dyed with differing concentrations of fluorescein to distinguish them from each other, while phycoerythrin is used as the label on the various labeled binding members used in the assay.

Still other examples of a differentiation parameter that can be used to distinguish among the various groups of particles are light scatter, or a combination of light scatter. Side angle light scatter varies with particle size, granularity, absorbance and surface roughness, while forward angle light scatter is mainly affected by size and refractive index. Thus, varying any of these qualities can serve as a means of distinguishing the various groups. Light emission can be varied by incorporating fluorescent materials in the microparticles and using fluorescent materials that have different fluorescence intensities or that emit fluorescence at different wavelengths, or by varying the amount of fluorescent material incorporated. By using fluorescence emissions at different wavelengths, the wavelength difference can be used to distinguish the particle groups from each other, while also distinguishing the labels in the labeled binding members from the labels that differentiate one particle group from another.

In a variation of the above, the microparticles will have two or more fluorochromes incorporated within them so that each microparticle in the array will have at least three differentiation parameters associated with it, i.e., light scatter together with fluorescent emissions at two separate wavelengths. For example, the microparticle can be made to contain a red fluorochrome such as Cy5 together with a far-red fluorochrome such as Cy5.5. Additional fluorochromes can be used to further expand the system. Each microparticle can thus contain a plurality of fluorescent dyes at varying wavelengths.

Still another example of a differentiation parameter that can be used to distinguish among the various groups of particles is absorbance. When light is applied to microparticles the absorbance of the light by the particles is indicated mostly by the strength of the laterally (side-angle) scattered light while the strength of the forward-scattered light is relatively unaffected. Consequently, the difference in absorbance between various colored dyes associated with the microparticles is determined by observing differences in the strength of the laterally scattered light.

A still further example of a differentiation parameter that can be used to distinguish among the various groups of particles is the number of particles in each group. The number of particles of each group is varied in a known way, and the count of particles having various assay responses is determined. The various responses are associated with a particular assay by the number of particles having each response.

As the above examples illustrate, a wide array of parameters or characteristics can be used as differentiation parameters to distinguish the microparticles of one group from those of another. The differentiation parameters may arise from particle size, from particle composition, from particle physical characteristics that affect light scattering, from excitable fluorescent dyes or colored dyes that impart different emission spectra and/or scattering characteristics to the microparticles, or from different concentrations of one or more fluorescent dyes. When the distinguishable microparticle parameter is a fluorescent dye or color, it can be coated on the surface of the microparticle, embedded in the microparticle, or bound to the molecules of the microparticle material. Thus, fluorescent microparticles can be manufactured by combining the polymer material with the fluorescent dye, or by impregnating the microparticle with the dye. Microparticles with dyes already incorporated and thereby suitable for use in the present invention are commercially available, from suppliers such as Spherotech, Inc. (Libertyville, Ill., USA) and Molecular Probes, Inc. (Eugene, Oreg., USA).

### VII. Reaction Mixtures

The present invention also provides for reaction mixtures used in the assays of the invention. Such mixtures comprise one or more of the components of the above-described method in the same aqueous reaction mixture, optionally in a mixture with a biological sample or a component thereof. In some embodiments, the reaction mixture comprises a biological sample from a human, and an anti-CA-125 capture agent (including but not limited to an antibody) and, in the same or parallel reaction mixture, capture agents, typically IGF2BP2, TGIF2LX, HMGA2, and or MYCN peptides, to detect IGF2BP2, TGIF2LX, HMGA2, and or MYCN autoantibodies. In some embodiments, the capture agents are linked to the same or different solid supports. In some embodiments, the solid support(s) is a bead. In some embodiments, the capture agents are detectably labeled. The reaction mixture can further include detection agents, optionally labeled. In some embodiments, the detection agent can be an anti-human IgG antibody.

Other possible components of the reaction mixture will be clear from the remainder of this document.

### VIII. Kits

The present invention also provides for kits of performing the methods of the invention as described herein and can include any combination of the reagents described herein. For example, the kit can comprise an anti-CA-125 capture agent (including but not limited to an antibody) and capture agents for IGF2BP2, TGIF2LX, HMGA2, and/or MYCN. In some embodiments, the capture agents will be linked to the same or different solid supports. In some embodiments, each capture reagent is like to a different solid support where the solid support is a bead. The kit can also include relevant detection agents for CA-125 or IGF2BP2, TGIF2LX, HMGA2, and/or MYCN autoantibodies. In some embodiments, the capture agents are detectably labeled.

In some embodiments, a kit of the invention further comprises one or more immunogenic fragments from IGF2BP2, TGIF2LX, HMGA2, and/or MYCN. In some embodiments, the fragments are linked to a solid support, e.g., a bead. The kits can further include a detection agent, optionally labeled or otherwise including a labeling reagent as well). The detection agent can be an antibody that specifically recognizes the antigen or can be an anti-human IgG antibody.

Other possible components of the kit will be clear from the remainder of this document.

### EXAMPLES

The following examples are offered to illustrate, but not to limit the claimed invention.

### Example

### Autoantibodies to IGF2BP2, TGIF2LX, HMGA2, and MYCN are Associated with Ovarian Cancer

### Patients

The study cohort consisted of 194 randomly selected woman of various ages with biopsy confirmed ovarian carcinoma. Multiple stages of ovarian cancer were included (stage I-III). Within this group 68 patients were either equivocal or negative for CA-125 based on the assay described below. The control group consisted of 154 female patients of various ages with no known ovarian cancer or other gynecological disease.

### CA-125 Assay

A capture antibody specific for CA-125 was coupled to Luminex beads. Antibody-coated beads were incubated with patient serum to allow any CA-125 present in the serum to bind. After washing away the serum, beads were incubated with a biotinylated detection antibody specific for CA-125. After removal of the detection antibody, beads were incubated with streptavidin conjugated phycoerythrin (PE). Following washing, beads were then passed through a Luminex detector (BioPlex 200) and the amount of bound PE, which is an indicator of the presence of CA-125, was quantified. Bound PE was reported as relative fluorescent intensity, which was converted to IU/mL of CA-125 using a standard curve included in the assay. Patients were then classified as CA-125 positive (>110 IU/mL), equivocal (35-109 IU/mL), or negative (<35 IU/mL).

### Peptide Assay for Autoantibodies

For each protein of interest peptides of 20 amino acids in length were designed and synthesized to regions of known reactivity in ovarian cancer patients based on prior epitope mapping experiments. These included three HMGA2 peptides (SEQ ID NOS:4-6), four IGFB2 peptides (SEQ ID NOS:7-10), six MYCN peptides (SEQ ID NOS:11-16), and five TGIF2LX peptides (SEQ ID NOS:17-21). Each of the 18 peptides was individually coupled to different Luminex beads, which each contain a unique dye identifier. All 18 bead types were mixed together and incubated with patient serum to allow any antibodies present in the serum to bind to the peptides. Following washing, beads were then incubated with PE conjugate goat anti-human antibody, which detects any antibodies bound to the peptides. After washing, the bead mixture was then passed through a Luminex detector (BioPlex 200). As each bead passes through the detector it is identified by its unique dye, which indicates which peptide is present, and the amount of bound PE is reported as relative fluorescent intensity. Ovarian cancer patients were considered positive for a given peptide if the relative fluorescent intensity was greater than the 98th percentile cutoff established using the control group.

### Protein Assay for Autoantibodies

Recombinant full-length his-tagged TGIF2LX, HMGA2, and IGF2BP2 proteins were expressed in HEK293 cells and purified using immobilized metal affinity chromatography (MYCN protein was not available for this study). Purified proteins were individually coupled to different Luminex beads, which each contain a unique identifier. The three bead types were mixed together and patient serum was assayed in the same manner as with the peptides. Ovarian cancer patients were considered positive for a given protein if the relative fluorescent intensity was greater than the 98th percentile cutoff established using the control group.

The results are shown in Table 1. The peptide sequences corresponding to the peptide number employed in Table 1 are provided in Table 2. Table 1 shows the number and percent-positive ovarian cancer patient with the cohort for each of the analytes (peptides and proteins). The threshold for positively is based on the values obtained with the peptides and protein in assaying the peptide and protein assay in the 154 normal individuals and determining the relative fluorescent intensity of the 98th percentile (2% false positives). Each analyte has its own threshold. The results demonstrated autoantibodies to these marker proteins were detected in ovarian cancer patients.

In the claims appended hereto, the term “a” or “an” is intended to mean “one or more.” The term “comprise” and variations thereof such as “comprises” and “comprising,” when preceding the recitation of a step or an element, are intended to mean that the addition of further steps or elements is optional and not excluded.

All patents, patent applications, and other published reference materials cited in this specification are hereby incorporated herein by reference in their entirety. Any discrepancy between any reference material cited herein or any prior art in general and an explicit teaching of this specification is intended to be resolved in favor of the teaching in this specification. This includes any discrepancy between an art-understood definition of a word or phrase and a definition explicitly provided in this specification of the same word or phrase.

### Full-Length Amino Acid Sequences of HMGA2, IGF2BP2, MYCN, and TGIF2LX:

```
HMGA2 (accession number NP_003475.1)
SEQ ID NO: 1
    1 MSARGEGAGQ PSTSAQGQPA APAPQKRGRG RPRKQQQEPT GEPSPKRPRG RPKGSKNKSP
  61 SKAAQKKAEA TGEKRPRGRP RKWDNLLPRT SSKKKTSLGN STKRSH
IGF2BP2 (accession number NP_006539.3)
SEQ ID NO: 2
    1 MMNKLYIGNL SPAVTADDLR QLFGDRKLPL AGQVLLKSGY AFVDYPDQNW AIRAIETLSG
  61 KVELHGKIME VDYSVSKKLR SRKIQIRNIP PHLQWEVLDG LLAQYGTVEN VEQVNTDTET
121 AVVNVTYATR EEAKIAMEKL SGHQFENYSF KISYIPDEEV SSPSPPQRAQ RGDHSSREQG
181 HAPGGTSQAR QIDFPLRILV PTQFVGAIIG KEGLTIKNIT KQTQSRVDIH RKENSGAAEK
241 PVTIHATPEG TSEACRMILE IMQKEADETK LAEEIPLKIL AHNGLVGRLI GKEGRNLKKI
301 EHETGTKITI SSLQDLSIYN PERTITVKGT VEACASAEIE IMKKLREAFE NDMLAVNQQA
361 NLIPGLNLSA LGIFSTGLSV LSPPAGPRGA PPAAPYHPFT THSGYFSSLY PHHQFGPFPH
421 HHSYPEQEIV NLFIPTQAVG AIIGKKGAHI KQLARFAGAS IKIAPAEGPD VSERMVIITG
481 PPEAQFKAQG RIFGKLKEEN FFNPKEEVKL EAHIRVPSST AGRVIGKGGK TVNELQNLTS
541 AEVIVPRDQT PDENEEVIVR IIGHFFASQT AQRKIREIVQ QVKQQEQKYP QGVASQRSK
MYCN (accession number NP_005369.2)
SEQ ID NO: 3
    1 MPSCSTSTMP GMICKNPDLE FDSLQPCFYP DEDDFYFGGP DSTPPGEDIW KKFELLPTPP
  61 LSPSRGFAEH SSEPPSWVTE MLLENELWGS PAEEDAFGLG GLGGLTPNPV ILQDCMWSGF
121 SAREKLERAV SEKLQHGRGP PTAGSTAQSP GAGAASPAGR GHGGAAGAGR AGAALPAELA
181 HPAAECVDPA VVFPFPVNKR EPAPVPAAPA SAPAAGPAVA SGAGIAAPAG APGVAPPRPG
241 GRQTSGGDHK ALSTSGEDTL SDSDDEDDEE EDEEEEIDVV TVEKRRSSSN TKAVTTFTIT
301 VRPKNAALGP GRAQSSELIL KRCLPIHQQH NYAAPSPYVE SEDAPPQKKI KSEASPRPLK
361 SVIPPKAKSL SPRNSDSEDS ERRRNHNILE RQRRNDLRSS FLTLRDHVPE LVKNEKAAKV
421 VILKKATEYV HSLQAEEHQL LLEKEKLQAR QQQLLKKIEH ARTC
TGIF2LX (accession number NP_620410.3)
SEQ ID NO: 4
    1 MEAAADGPAE TQSPVEKDSP AKTQSPAQDT SIMSRNNADT GRVLALPEHK KKRKGNLPAE
  61 SVKILRDWMY KHRFKAYPSE EEKQMLSEKT NLSLLQISNW FINARRRILP DMLQQRRNDP
121 IIGHKTGKDA HATHLQSTEA SVPAKSGPSG PDNVQSLPLW PLPKGQMSRE KQPDPESAPS
181 QKLTGIAQPK KKVKVSVTSP SSPELVSPEE HADFSSFLLL VDAAVQRAAE LELEKKQEPN
241 P
```

TABLE 1
For each analyte, numbers indicate number of positive patients for each category (i.e. all patients or
CA125 <110 IU/mL). Percentages indicate percent of positive patients out of total in each category. Cutoff
for positivity is the 98^{th} percentile based on running the same analytes in 154 control samples.

| | HMGA2 | IGF2BP2 | TGIF2LX | HMGA2 | HMGA2 | HMGA2 | IGF2BP2 | IGF2BP2 | IGF2BP2 | IGF2BP2 | TGIF2LX |
|---|---|---|---|---|---|---|---|---|---|---|---|
| | Protein | Protein | Protein | Pep788 | Pep789 | Pep793 | Pep798 | Pep804 | Pep809 | Pep823 | Pep847 |
| All OvCa | 16 | 8 | 10 | 10 | 7 | 10 | 1 | 14 | 4 | 7 | 9 |
| (n = 194) | | | | | | | | | | | |
| CA125 <110 | 5 | 0 | 2 | 6 | 2 | 2 | 1 | 3 | 2 | 1 | 5 |
| (n = 68) | | | | | | | | | | | |
| All OvCa | 8.2% | 4.1% | 5.2% | 5.2% | 3.6% | 5.2% | 0.5% | 7.2% | 2.1% | 3.6% | 4.6% |
| (n = 194) | | | | | | | | | | | |
| CA125 <110 | 7.4% | 0.0% | 2.9% | 8.8% | 2.9% | 2.9% | 1.5% | 4.4% | 2.9% | 1.5% | 7.4% |
| (n = 68) | | | | | | | | | | | |

| | TGIF2LX | TGIF2LX | TGIF2LX | TGIF2LX | MYCN | MYCN | MYCN | MYCN | MYCN | MYCN |
|---|---|---|---|---|---|---|---|---|---|---|
| | Pep852 | Pep855 | Pep856 | Pep858 | Pep863 | Pep867 | Pep875 | Pep877 | Pep889 | Pep897 |
| All OvCa | 10 | 6 | 2 | 20 | 4 | 1 | 2 | 15 | 3 | 6 |
| (n = 194) | | | | | | | | | | |
| CA125 <110 | 4 | 2 | 0 | 4 | 1 | 0 | 1 | 8 | 1 | 1 |
| (n = 68) | | | | | | | | | | |
| All OvCa | 5.2% | 3.1% | 1.0% | 10.3% | 2.1% | 0.5% | 1.0% | 7.7% | 1.5% | 3.1% |
| (n = 194) | | | | | | | | | | |
| CA125 <110 | 5.9% | 2.9% | 0.0% | 5.9% | 1.5% | 0.0% | 1.5% | 11.8% | 1.5% | 1.5% |
| (n = 68) | | | | | | | | | | |

TABLE 2
Peptide fragments of Table 1

| | | |
|---|---|---|
| Pep788 | MSARGEGAGQPSTSAQGQPA | SEQ ID NO:5 |
| Pep789 | SAQGQPAAPAPQKRGRGRPR | SEQ ID NO:6 |
| Pep793 | KKAEATGEKRPRGRPRKWDN | SEQ ID NO:7 |
| Pep798 | MMNKLYIGNLSPAVTADDLR | SEQ ID NO:8 |
| Pep804 | LRSRKIQIRNIPPHLQWEVL | SEQ ID NO:9 |
| Pep809 | QFENYSFKISYIPDEEVSSP | SEQ ID NO:10 |
| Pep823 | TVKGTVEACASAEIEIMKKL | SEQ ID NO:11 |
| Pep847 | TGRVLALPEHKKKRKGNLPA | SEQ ID NO:18 |
| Pep852 | RRRILPDMLQQRRNDPIIGH | SEQ ID NO:19 |
| Pep855 | AKSGPSGPDNVQSLPLWPLP | SEQ ID NO:20 |
| Pep856 | LPLWPLPKGQMSREKQPDPE | SEQ ID NO:21 |
| Pep858 | LTGIAQPKKKVKVSVTSPSS | SEQ ID NO:22 |
| Pep863 | CKNPDLEFDSLQPCFYPDED | SEQ ID NO:12 |
| Pep867 | GFAEHSSEPPSWVTEMLLEN | SEQ ID NO:13 |
| Pep875 | RAGAALPAELAHPAAECVDP | SEQ ID NO:14 |
| Pep877 | PVNKREPAPVPAAPASAPAA | SEQ ID NO:15 |
| Pep889 | SEASPRPLKSVIPPKAKSLS | SEQ ID NO:16 |
| Pep897 | EKLQARQQQLLKKIEHARTC | SEQ ID NO:17 |

What is claimed is:

1. A method of determining the amount of autoantibodies to insulin-like growth factor binding protein 2 (IGF2BP2), HMGA2, MYCN, and TGIF2LX in a blood sample from a human patient that is being evaluated for ovarian cancer, the method comprising,
contacting a blood sample obtained from the patient with one or more peptides of 10 amino acids or greater in length from each of the antigens IGF2BP2, HMGA2, MYCN, and TGIF2LX; and
measuring the amount of selective binding of autoantibodies to the one or more peptides.

2. The method of claim 1, further comprising measuring the level of CA-125 in a blood sample from the patient.

3. The method of claim 1, wherein the peptides are attached to a solid support.

4. The method of claim 3, wherein the peptides comprise peptides having the sequences of SEQ ID NOS:1-22.

5. The method of claim 3, wherein the peptides are linked to the same solid support.

6. The method of claim 3, wherein each peptide is linked to a different solid support.

7. The method of claim 6, wherein each peptide is linked to a different bead, wherein each bead is distinguishable by flow cytometry.

8. The method of claim 3, wherein the peptides from each antigen are linked to the same solid support and the peptide from the different antigens are linked to different solid supports.

9. The method of claim 1, wherein specific binding of the autoantibody to the one or more peptides is detected by detecting binding of an anti-human IgG antibody to the autoantibody.

10. The method of claim 2, further comprising administering an agent to treat ovarian cancer when the patient has:
a CA-125 level over about 100 IU/mL; or
a CA-125 level between about 35 and 100 IU/mL and an autoantibody that specifically binds to IGF2BP2, HMGA2, MYCN, or TGIF2LX, or a fragment thereof; or
a negative CA-125 result and an autoantibody that specifically binds to IGF2BP2, HMGA2, MYCN, or TGIF2LX, or a fragment thereof.

11. A kit for detecting ovarian cancer in a human patient, wherein the kit comprises the following components:
one or more IGF2BP2 peptides that specifically detect an autoantibody to IGF2BP2;
one or more HMGA2 peptides that specifically detect an autoantibody to HMGA2;
one or more TGIF2LX peptides that specifically detect an autoantibody to TGIF2LX; and
one or more MYCN peptides that specifically detect an autoantibody to MYCN.

12. The kit of claim 11, further comprising an anti-CA-125 antibody component.

13. The kit of claim 11, wherein the kit comprises:
an IGF2B2 protein comprising the IGF2B2 amino acid sequence set forth in SEQ ID NO:2,
an IGF2B2 peptide comprising the amino acid sequence set forth in SEQ ID NO:8,
an IGF2B2 peptide comprising the amino acid sequence set forth in SEQ ID NO:9,
an IGF2B2 peptide comprising the amino acid sequence set forth in SEQ ID NO:10 and
an IGF2B2 peptide comprising the amino acid sequence set forth in SEQ ID NO:11.

14. The kit of claim 11, wherein the kit comprises:
an HMGA2 protein comprising the HMGA2 amino acid sequence set forth in SEQ ID NO:1,
an HMGA2 peptide comprising the amino acid sequence set forth in SEQ ID NO:5,
an HMGA2 peptide comprising the amino acid sequence set forth in SEQ ID NO:6, and
an HMGA2 peptide comprising the amino acid set forth in SEQ ID NO:7.

15. The kit of claim 11, wherein the kit comprises:
a MYCN protein comprising the MYCN amino acid sequence set forth in SEQ ID NO:3,
a MYCN peptide comprising the amino acid sequence set forth in SEQ ID NO:12,
a MYCN peptide comprising the amino acid sequence set forth in SEQ ID NO:13,
a MYCN peptide comprising the amino acid sequence set forth in SEQ ID NO:14,
a MYCN peptide comprising the amino acid sequence set forth in SEQ ID NO:15,
a MYCN peptide comprising the amino acid sequence set forth in SEQ ID NO:16, and
a MYCN peptide comprising the amino acid sequence set forth in SEQ ID NO:17.

16. The kit of claim 11, wherein the kit comprises:
a TGIF2LX protein comprising the TGIF2LX amino acid sequence set forth in SEQ ID NO:4;
a TGIF2LX peptide comprising the amino acid sequence set forth in SEQ ID NO:18;
a TGIF2LX peptide comprising the amino acid sequence set forth in SEQ ID NO:19;
a TGIF2LX peptide comprising the amino acid sequence set forth in SEQ ID NO:20;
a TGIF2LX peptide comprising the amino acid sequence set forth in SEQ ID NO:21; and
a TGIF2LX peptide comprising the amino acid sequence set forth in SEQ ID NO:22.

17. The kit of claim 11, wherein the components are linked to a solid support.

18. The kit of claim 17, wherein the components are individually linked to a different solid support.

19. The kit of claim 18, wherein the solid supports are beads.

20. The kit of claim 19, wherein the beads to which the components are individually linked are distinguishable from one another by flow cytometry.

21. The kit of claim 11, wherein the components are linked to the same solid support.

22. The kit of claim 11, further comprising an anti-human IgG antibody.

23. The kit of claim 22, wherein the anti-human IgG antibody is linked to a detectable label.


SEQUENCE LISTING (22 sequences)

SEQ ID NO: 1 - protein, 106 aa, Homo sapiens
  high mobility group AT-hook protein 2 (HMGA2), high mobility group protein HMGI-C (HMGI-C) isoform b, HMG nonhistone chromosomal protein isoform I-C, BABL, LIPO, STQT29
MSARGEGAGQPSTSAQGQPAAPAPQKRGRGRPRKQQQEPTGEPSPKRPRGRPKGSKNKSPSKAAQKKAEATGEKRPRGRPRKWDNLLPRTSSKKKTSLGNSTKRSH

SEQ ID NO: 2 - protein, 599 aa, Homo sapiens
  insulin-like growth factor 2 mRNA-binding protein 2 (IGF2BP2) isoform a, VICKZ family member 2 (VICKZ2), hepatocellular carcinoma autoantigen p62, IMP-2
MMNKLYIGNLSPAVTADDLRQLFGDRKLPLAGQVLLKSGYAFVDYPDQNWAIRAIETLSGKVELHGKIMEVDYSVSKKLRSRKIQIRNIPPHLQWEVLDGLLAQYGTVENVEQVNTDTETAVVNVTYATREEAKIAMEKLSGHQFENYSFKISYIPDEEVSSPSPPQRAQRGDHSSREQGHAPGGTSQARQIDFPLRILVPTQFVGAIIGKEGLTIKNITKQTQSRVDIHRKENSGAAEKPVTIHATPEGTSEACRMILEIMQKEADETKLAEEIPLKILAHNGLVGRLIGKEGRNLKKIEHETGTKITISSLQDLSIYNPERTITVKGTVEACASAEIEIMKKLREAFENDMLAVNQQANLIPGLNLSALGIFSTGLSVLSPPAGPRGAPPAAPYHPFTTHSGYFSSLYPHHQFGPFPHHHSYPEQEIVNLFIPTQAVGAIIGKKGAHIKQLARFAGASIKIAPAEGPDVSERMVIITGPPEAQFKAQGRIFGKLKEENFFNPKEEVKLEAHIRVPSSTAGRVIGKGGKTVNELQNLTSAEVIVPRDQTPDENEEVIVRIIGHFFASQTAQRKIREIVQQVKQQEQKYPQGVASQRSK

SEQ ID NO: 3 - protein, 464 aa, Homo sapiens
  N-myc proto-oncogene protein (MYCN, N-myc, NMYC), v-myc avian myelocytomatosis viral relared oncogene, neuroblastoma derived homolog, class E basic helix-loop-helix protein 37 (bHLHe37), neuroblastoma MYC oncogene, MODED, ODED, MYCNOT
MPSCSTSTMPGMICKNPDLEFDSLQPCFYPDEDDFYFGGPDSTPPGEDIWKKFELLPTPPLSPSRGFAEHSSEPPSWVTEMLLENELWGSPAEEDAFGLGGLGGLTPNPVILQDCMWSGFSAREKLERAVSEKLQHGRGPPTAGSTAQSPGAGAASPAGRGHGGAAGAGRAGAALPAELAHPAAECVDPAVVFPFPVNKREPAPVPAAPASAPAAGPAVASGAGIAAPAGAPGVAPPRPGGRQTSGGDHKALSTSGEDTLSDSDDEDDEEEDEEEEIDVVTVEKRRSSSNTKAVTTFTITVRPKNAALGPGRAQSSELILKRCLPIHQQHNYAAPSPYVESEDAPPQKKIKSEASPRPLKSVIPPKAKSLSPRNSDSEDSERRRNHNILERQRRNDLRSSFLTLRDHVPELVKNEKAAKVVILKKATEYVHSLQAEEHQLLLEKEKLQARQQQLLKKIEHARTC

SEQ ID NO: 4 - protein, 241 aa, Homo sapiens
  TGFB-induced factor 2-like, X-linked (TGIF2LX, TGIFLX), homeobox protein TGIF2LX, TGIF-like on the X, TGF-beta-induced transcription factor 2-like protein
MEAAADGPAETQSPVEKDSPAKTQSPAQDTSIMSRNNADTGRVLALPEHKKKRKGNLPAESVKILRDWMYKHRFKAYPSEEEKQMLSEKTNLSLLQISNWFINARRRILPDMLQQRRNDPIIGHKTGKDAHATHLQSTEASVPAKSGPSGPDNVQSLPLWPLPKGQMSREKQPDPESAPSQKLTGIAQPKKKVKVSVTSPSSPELVSPEEHADFSSFLLLVDAAVQRAAELELEKKQEPNP

SEQ ID NO: 5 - protein, 20 aa, Artificial Sequence
  synthetic HMGA2 peptide fragment Pep788
MSARGEGAGQPSTSAQGQPA

SEQ ID NO: 6 - protein, 20 aa, Artificial Sequence
  synthetic HMGA2 peptide fragment Pep789
SAQGQPAAPAPQKRGRGRPR

SEQ ID NO: 7 - protein, 20 aa, Artificial Sequence
  synthetic HMGA2 peptide fragment Pep793
KKAEATGEKRPRGRPRKWDN

SEQ ID NO: 8 - protein, 20 aa, Artificial Sequence
  synthetic IGF2BP2 peptide fragment Pep798
MMNKLYIGNLSPAVTADDLR

SEQ ID NO: 9 - protein, 20 aa, Artificial Sequence
  synthetic IGF2BP2 peptide fragment Pep804
LRSRKIQIRNIPPHLQWEVL

SEQ ID NO: 10 - protein, 20 aa, Artificial Sequence
  synthetic IGF2BP2 peptide fragment Pep809
QFENYSFKISYIPDEEVSSP

SEQ ID NO: 11 - protein, 20 aa, Artificial Sequence
  synthetic IGF2BP2 peptide fragment Pep823
TVKGTVEACASAEIEIMKKL

SEQ ID NO: 12 - protein, 20 aa, Artificial Sequence
  synthetic MYCN peptide fragment Pep863
CKNPDLEFDSLQPCFYPDED

SEQ ID NO: 13 - protein, 20 aa, Artificial Sequence
  synthetic MYCN peptide fragment Pep867
GFAEHSSEPPSWVTEMLLEN

SEQ ID NO: 14 - protein, 20 aa, Artificial Sequence
  synthetic MYCN peptide fragment Pep875
RAGAALPAELAHPAAECVDP

SEQ ID NO: 15 - protein, 20 aa, Artificial Sequence
  synthetic MYCN peptide fragment Pep877
PVNKREPAPVPAAPASAPAA

SEQ ID NO: 16 - protein, 20 aa, Artificial Sequence
  synthetic MYCN peptide fragment Pep889
SEASPRPLKSVIPPKAKSLS

SEQ ID NO: 17 - protein, 20 aa, Artificial Sequence
  synthetic MYCN peptide fragment Pep897
EKLQARQQQLLKKIEHARTC

SEQ ID NO: 18 - protein, 20 aa, Artificial Sequence
  synthetic TGIF2LX peptide fragment Pep847
TGRVLALPEHKKKRKGNLPA

SEQ ID NO: 19 - protein, 20 aa, Artificial Sequence
  synthetic TGIF2LX peptide fragment Pep852
RRRILPDMLQQRRNDPIIGH

SEQ ID NO: 20 - protein, 20 aa, Artificial Sequence
  synthetic TGIF2LX peptide fragment Pep855
AKSGPSGPDNVQSLPLWPLP

SEQ ID NO: 21 - protein, 20 aa, Artificial Sequence
  synthetic TGIF2LX peptide fragment Pep856
LPLWPLPKGQMSREKQPDPE

SEQ ID NO: 22 - protein, 20 aa, Artificial Sequence
  synthetic TGIF2LX peptide fragment Pep858
LTGIAQPKKKVKVSVTSPSS